(19) United States
(12) Patent Application Publication
(10) Pub. No.: US 2008/0268521 A1
(43) Pub. Date: Oct. 30, 2008

(54) Purification of Coagulation Factor VII Polypeptides

(75) Inventor: Haleh Ahmadian, Solrod Strand (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 12/064,901

(22) PCT Filed: Sep. 1, 2006
(86) PCT No.: PCT/EP2006/065929

§ 371 Date: Jun. 24, 2008

(30) Foreign Application Priority Data
Sep. 1, 2005 (EP) 05107991.1

(51) Int. Cl. C12N 9/50

(52) U.S. Cl. 435/212

(57) ABSTRACT

An improved method for producing FVII and FVIIa polypeptides is disclosed. Also provided are FVII and FVIIa compositions having low contents of auto-degradation products.

### FIELD OF THE INVENTION

The present invention relates to the field of protein purification. In particular, the invention relates to a method for purification of blood coagulation Factors VII and VIIa.

### BACKGROUND OF THE INVENTION

Factor VII (FVII) is a trace plasma glycoprotein that circulates in blood as a single-chain zymogen. The zymogen is catalytically inactive. Single-chain Factor VII may be converted into catalytically active two-chain Factor VIIa (FVIIa) by cleavage of the internal Arg₁₅₂-Ile₁₅₃ peptide bond. This conversion of zymogen Factor VII into the activated two-chain Factor VIIa is catalysed in vitro by Factor Xa, Factor XIIa, Factor IXa, Factor VIIa, kallikrein and thrombin. Factor Xa is believed to be the major physiological activator of Factor VII.

Activated Factor VII (FVIIa) is widely used as a therapeutic protein for the treatment of various coagulation disorders that may be caused by clotting factor deficiencies or clotting factor inhibitors. FVIIa has also been used to control excessive bleeding occurring in subjects with a normally functioning blood clotting cascade. Such bleeding may for example be caused by a defective platelet function, thrombocytopenia, von Willebrand's disease as well as during surgery and other forms of tissue damage.

FVIIa is a serine protease and its hydrolysis of the FVII sequence does not necessarily stop after auto-activation by cleavage of the Arg₁₅₂ bond. Other bonds adjacent to basic amino acids in the FVII sequence which are also subject to FVIIa mediated hydrolysis is at Lys₃₈-Leu₃₉, Arg₂₉₀-Gly₂₉₁ and Arg₃₁₅-Lys₃₁₆. Cleavage of FVII polypeptide by FVIIa at other sites than Arg₁₅₂-Ile₁₅₃ is known as auto-degradation. A good correlation has been shown between the degree of activation of FVII and the amount of auto-degraded products (Mollerup et al. Biotech & bioeng. 48, 1995, 501-505). Thus, it is important to limit activation of FVII polypeptides during purification process. During the last >25 years a key element in methods for purification of FVII has been anion exchange chromatography (Flengsrud, Eur. 3. Biochem. 98, 1979, 455-464, U.S. Pat. No. 4,637,932 B1, and Jurlander et al. Sem. Thromb. Hemost. 27(4), 2001, 373-383). Since it is well known that FVII is auto-activated in the presence of positively charged surfaces such as an anion exchange matrix (Pedersen et al., Biochem. 28(24), 1989, 9331-6), it has been necessary to perform the purification under conditions allowing auto-activation but at the same time limiting auto-degradation during the ion-exchange purification.

One approach to overcome this auto-activation and auto-degradation of FVII during ion-exchange purification has been addition of FVIIa inhibitors such as benzamidine, soybean trypsin inhibitor, and phenyl-methyl-sulfonyl fluoride. However, these compounds also represent drawbacks and they must also be eliminated from the FVIIa drug substance.

Another approach to overcome the auto-activation and auto-degradation of FVII during ion-exchange purification has been cooling of the entire purification equipment and solutions to low temperatures such as 0-10° C., an ordinary procedure during prolonged handling of proteins labile to enzymatic or microbial degradation. Cooling of process chromatographic equipment is, however, cumbersome and expensive. Alternatively, the entire purification process has to be performed in cold rooms resulting in expensive manufacturing facilities.

U.S. Pat. No. 6,777,390 (Baxter) concerns purification of factor VII from cryosupernatant by AIEX and subsequent hydrophobic chromatography on Phenyl-Sepharose.

U.S. Pat. No. 4,637,932 (Miles Lab.) concerns a process for purification of a concentrate containing Factors VII and VIIa.

WO 2004/083421 describes purification of solutions of FVII polypeptides at certain pH intervals and Ca²⁺ concentrations in order to reduce the activity of FVII polypeptides.

US 20010007901 A1 relates to purification of FVII and FVIIa by affinity chromatography using immobilized soluble thromboplastin (i.e. tissue factor).

Thermal effects on an enzymatically latent conformation of FVIIa have been disclosed in Pedersen et al., Eur. 3. Biochem. 261, 1999, 124-129 and in Freskgard et al. Biochemistry, 37, 1998, 7203-7212.

The article “Amino acid sequence and posttranslational modifications of human factor VIIa from plasma and transfected baby hamster kidney cells”, Biochemistry, 1988 Oct. 4; 27(20):7785-93, reports that some heavy chain degradation products co-purify with intact activated Factor VII.

The article “FVIIa derivatives obtained by autolytic and controlled cathepsin G mediated cleavage”, FEBS Lett. 1993 Feb. 15; 317(3):245-9), states that heavy chain cleaved forms of Factor VII cannot be isolated from Factor VII under non-denaturing conditions.

Thus, there is a need for improved purification processes for the manufacture of FVII and FVIIa, such as methods for purification of Factor VII polypeptides without the need for cooling the equipment or cold rooms and/or purification method for Factor VII polypeptides resulting in low content of Factor VII auto-degradation products.

### SUMMARY OF THE INVENTION

The present invention provides a method for purification of Factor VII polypeptides while limiting or even avoiding auto-activation and/or auto-degradation. In a first aspect the invention provides a method for purification of a Factor VII polypeptide wherein the temperature during purification is in the range from about 30° C. to about 50° C. In a second aspect the invention relates to a process for purification of a Factor VII polypeptide wherein the temperature during at least one purification step is in the range from about 30° C. to about 50, such as, e.g., about 30° C. to about 45° C., or about 35° C. to about 45° C. In one embodiment the temperature is in the range from about 35° C. to about 45° C.

In one embodiment of the present invention, the purification of Factor VII polypeptides is performed in an aqueous solution comprising an organic modifier. In one embodiment, the organic modifier is ethanol. In yet another embodiment, the purification of FVII polypeptides is performed using protein stabilizers selected from a group of sugars such as sucrose and/or amino acids such as arginine.

In yet another embodiment, the purification of Factor VII polypeptides is performed using refolding agents such as, e.g., ethylene glycol.

In another aspect the invention provides a chromatographic purification of Factor VII polypeptides wherein the temperature during purification is in the range from about 35° C. to about 45° C. In one embodiment the chromatographic purification is ion exchange purification (IEC). In another embodiment the chromatographic purification is anion exchange purification (AIEC). In another embodiment the chromatographic purification is hydrophobic interaction chromatography (HIC).

In one aspect, the invention provides a method for preventing activation during purification of Factor VII polypeptides, the method comprising the step:
a) the temperature during purification is in the range from about 30° C. to about 50° C.

In one embodiment thereof, the Factor VII polypeptides are non-activated Factor VII polypeptides or a mixture of non-activated and activated Factor VII polypeptides.

In one aspect, the invention provides a method for preventing formation of auto-degradation products during purification of activated Factor VII polypeptides, the method comprising the steps:
a) the temperature during purification is in the range from about 30° C. to about 50° C.

In one embodiment thereof, the Factor VII polypeptides are activated Factor VII polypeptides.

In another aspect the present invention provides an activated Factor VII polypeptide (Factor VIIa polypeptide) product manufactured by a process comprising the steps:

a) purifying a Factor VII polypeptide using the method according to the present invention, and
b) activating the purified Factor VII polypeptide of step a) to the corresponding activated Factor VII polypeptide, and
c) isolating the activated Factor VII polypeptide of step b) to give the resulting Factor VIIa polypeptide product, which has retained at least 30% of its specific activity.

In another aspect the present invention provides a Factor VII polypeptide product manufactured by a process comprising the steps:

a) purifying the Factor VII polypeptide using the method according to the present invention, and
b) isolating the Factor VII polypeptide of step a) to give the resulting Factor VII polypeptide product, which has retained at least 30% of its specific activity.

In yet another aspect the present invention provides an activated Factor VII polypeptide product manufactured by a process comprising the steps:

a) purifying the activated Factor VII polypeptide using the method according to the present invention, and
b) isolating the activated Factor VII polypeptide of step a) to give the resulting activated Factor VII polypeptide product, which has retained at least 30% of its specific activity.

In another aspect the present invention provides a pharmaceutical composition comprising a Factor VII polypeptide, and containing less than 15% (w/w) (i.e., w auto-degradation products/w protein) auto-degradation products produced by cleavage at any of positions 38, 290, 315 or combinations thereof in said Factor VII polypeptide. In one embodiment the composition contains less than 5% (w/w protein) degradation products produced by cleavage at any of positions 38, 290, 315 or combinations thereof.

### BRIEF DESCRIPTION OF THE DRAWINGS

FIG. 1 shows the amino acid sequence of native human coagulation Factor VII (FVII).

### DETAILED DESCRIPTION OF THE INVENTION

It has been found that FVII polypeptides can be purified at a temperature in the range from about 30° C. to about 50° C., such as, e.g., about 30° C. to about 45° C. or about 35° C. to about 45° C. or about 35° C. to about 50° C., while retaining biological activity.

Purification at temperatures 30-50° C. reduces premature activation of Factor VII polypeptides. Furthermore, using the method of the invention, it is possible to purify activated Factor VII polypeptides without increasing the content of auto-degraded products.

The term “GLA” as used herein means 4-carboxyglutamic acid (γ-carboxyglutamate). Non-limiting examples of modifications of amino acid residues include amidation, alkylation, acylation and pegylation.

Factor VII polypeptide typically originates from an industrial-scale production process, e.g. a cell culture, a cloned animal (e.g. cows, pigs, sheep, goats, and fish) or insect, or the like, in particular from a cell culture.

FVII polypeptides might be partly purified prior to purification at elevated temperatures.

The term “polypeptide” as used herein means a compound comprising at least five constituent amino acid residues covalently connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxy-glutamic acid, ornithine, phosphoserine, D-alanine, D-glutamic acid. Synthetic amino acids comprise amino acids manufactured by organic synthesis, e.g. D-isomers of the amino acids encoded by the genetic code and Aib α-aminoisobutyric acid), Abu (α-aminobutyric acid), Tle (tert-butylglycine), and β-alanine. A polypeptide may comprise a single peptide chain or it may comprise more than one polypeptide chain, such as FVII being a single chain and FVIIa being two chains connected by disulphide bonds.

Inactivation of the Factor VII polypeptide by high temperatures may also be augmented by the addition of an organic modifier to the solutions used for the purification. The term “organic modifier” as used herein refers to an organic compound which is added to aqueous solutions in order improve the properties of the solution. Organic modifiers may be monohydric alcohols, polyhydric alcohols as well as nitriles and ketones. Non-limiting examples of organic modifiers are methanol, ethanol, 1-propanol, 2-propanol, t-butanol, hexylene glycol (4-methyl-2,4-pentanediol), neopentyl alcohol (2,2-dimethyl-1,3-propanediol), acetonitrile, and acetone. The concentration of the organic modifier is from about 2% w/w to about 40% w/w, such as e.g. from about 2% w/w to about 10% w/w.

In another embodiment, the purification of Factor VII polypeptides is performed using protein stabilizers selected from a group of sugars such as sucrose and/or amino acids such as arginine. In one embodiment the protein stabilizer is arginine in a concentration from about 0.5M to about 5M.

In yet another embodiment, the purification of Factor VII polypeptides is performed using refolding agents such as, e.g., ethylene glycol. In one embodiment ethylene glycol is used as refolding agent in a concentration from about 0.5M to about 10M.

The purification method according to the present invention may be chromatographic purification, membrane purification, or another purification method which is compatible with increased temperatures. In one embodiment the purification is a chromatographic purification. A widely used chromatographic purification method is ion exchange chromatography, such as anion exchange chromatography (AIEC). Commonly used anion exchange resins are Q-resin, a Quaternary amine, and DEAE resin, DiEthylAminoEthane. Anion exchange resins are commercially available, e.g. Mono Q (Amersham Biosciences), Source 15Q or 30Q (Amersham Biosciences), Poros 20HQ or 50HQ (Perceptive Biosystems), Toyopearl Q650S (Toso Haas) and others.

Cation exchangers can also be used for the purification of Factor VII polypeptides. The most widely used cation exchangers contain a carboxymethyl (CM) or sulfopropyl (SP) group. Examples of such cation exchangers include Toyopearl CM-650 or Toyopearl SP-650 (Toso Haas), Source 15 S or 30 S, CM or SP Sepharose Fast Flow (Amersham Biosciences).

In another embodiment the chromatographic purification is selected from the group consisting of hydrophobic interaction chromatography, size exclusion chromatography, and affinity chromatography.

Hydrophobic interaction chromatography (HIC) is a separation method that takes advantage of the hydrophobic properties of the proteins. The adsorption is promoted by the hydrophobic interactions between non-polar regions on the protein and immobilized hydrophobic ligands on a solid support. Adsorption is achieved at high salt concentrations in the aqueous mobile phase and elution is facilitated by decreasing the salt concentration. The hydrophobic interaction chromatography material is a matrix substituted with hydrophobic ligands suchs as ethyl-, butyl-, phenyl or hexyl-groups. Preferred material is a matrix substituted with a butyl or a phenyl ligand.

For performing size exclusion chromatography, the gel is typically selected from the group of polymeric gels including dextran-based gels such as Sephadex, agarose based gels such as Sepharose, polyacrylamide gels such as Sephacryl and composite gels pre-pared from two kind of gels such as Superdex.

A number of affinity chromatographic purification methods have been used in the purification of Factor VII polypeptides, e.g. using an anti-Factor VII antibody column (see, e.g., Wakabayashi et al., J. Biol. Chem. 261:11097, 1986; and Thim et al., Biochem. 27:7785, 1988), using immobilized soluble thromboplastin i.e. tissue factor (US 20010007901 A1), and using immobilized triazine ligands (WO 97/10887).

In one embodiment, the method for purification of a FVII polypeptide, wherein the temperature is in the range from about 30° C. to about 50° C., such as, e.g., from about 30° C. to about 45° C., is affinity chromatography applying a triazine ligand. The triazine ligands disclosed in WO 97/10887 are incorporated by reference. In another embodiment, the affinity chromatography uses an immobilized antibody against a Factor FVII polypeptide.

During chromatographic purification of a Factor VII polypeptide it is often desirable to perform the chromatography using solutions comprising buffers, salts etc. In one embodiment the solution for elution of the FVII polypeptide comprises a chelating agent. In another embodiment the chelating agent is selected from citrate, oxalate, tartrate, nitrilotriacetic acid (NTA), ethylenediaminetetraacetic acid (EDTA) and, (ethylenebis(oxyethylenenitrilo)) tetraacetic acid EGTA). A list of buffers that can be used is given in Buffer solutions, The basics, Beynon R. J. & Easterby J. S., Oxford University Press, 1996. Examples of buffers that can be used for equilibration, washing, elution or in load solution comprise glycylglycine, histidine, tris(hydroxymethyl) aminomethane (TRIS), 3-(N-Morpholino)-propanesulfonic acid (MOPS), 2-Morpholinoethanesulfonic acid (MES) and citrate. In another embodiment the solution used for load or elution has a pH in the range from about 5.0 to about 9.5. In another embodiment the solution used for elution comprises Ca²⁺.

Another purification method which is encompassed in the present invention is membrane purification. In one embodiment the membrane purification is microfiltration. In another embodiment the membrane filtration is sterile filtration, e.g. using a 0.2 μm filter. In another embodiment the membrane filtration is performed by ultrafiltration. In another embodiment the ultrafiltration is performed using a membrane having a molecular cut off value of approximately 30 kD. In another embodiment the ultrafiltration comprises a diafiltration step.

In one embodiment the temperature used during the purification process is in the range from about 35° C. to about 45° C. In another embodiment the temperature used during the purification process is in the range from about 35° C. to about 40° C.; in another embodiment the temperature used during the purification process is in the range from about 40° C. to about 45° C.; in another embodiment the temperature used during the purification process is in the range from about 35° C. to about 45° C.; in another embodiment the temperature used during the purification process is in the range from about 40° C. to about 50° C.

In another embodiment the purification is performed with a temperature in the range from about 35° C. to about 45° C. and with a concentration of ethanol in the range from about 2% w/w to about 40% w/w. In another embodiment the purification is performed with a temperature in the range from about 30° C. to about 45° C. and with a concentration of ethanol in the range from about 10% w/w to about 40% w/w. In another embodiment the purification is performed with a temperature in the range from about 30° C. to about 45° C. and with a concentration of ethanol in the range from about 2% w/w to about 20% w/w. In another embodiment the purification method is anion exchange chromatography performed with aqueous solutions comprising ethanol in a concentration from about 2% w/w to about 20% w/w at a temperature in the range from about 35° C. to about 45° C.

Following the purification at elevated temperatures (where the Factor VII polypeptide presumably attains a conformation with little proteolytic activity) it may be expedient to control the condition under which the polypeptide refolds when temperature is lowered.

In one embodiment the elution is performed into a solution comprising arginine or/and ethylene glycol. In another embodiment arginine or/and ethylene glycol is added to the elution buffer. The concentration of arginine is varied between about 0.5M to about 5M and the concentration of ethylene glycol is from about 0.5M to about 10M.

In one embodiment, the resulting Factor VII polypeptide retains at least 30% of its specific activity compared to the Factor VII polypeptide prior to purification by the method of the present invention. In another embodiment, the resulting Factor VII polypeptide retains at least 40% of its specific activity, such as e.g. at least 60% or at least 80%.

When purifying non-activated (zymogen or single-chain) Factor VII polypeptides, including mixtures of non-activated and activated forms:

The term “retaining x % of its specific activity” is intended to mean that the Factor VII polypeptide after the elevated temperature purification step according to the present invention—and after a subsequent activation step—exhibits at least x % of the specific activity as measured in one or more of the assays described herein compared to the specific activity of a reference preparation purified—and activated—under the same conditions except for the temperature during the purification step being 0° C.-9° C. (and not 30° C.-50° C.). A reference preparation refers to a preparation comprising a polypeptide that is identical to that purified according to the present invention (such as, e.g., wild-type Factor VII or a particular variant or derivative).

### When Purifying Activated (Two-Chain) Factor Vii Polypeptides:

The term “retaining x % of its specific activity” is intended to mean that the activated Factor VII polypeptide after the elevated temperature purification step according to the present invention exhibits at least x % of the specific activity as measured in one or more of the assays described herein compared to the specific activity of the Factor VII polypeptide before said purification step.

In a preferred embodiment of the present invention, the content of auto-degradation products, produced by cleavage at any of positions 38, 290, 315 or combinations thereof in the Factor VII polypeptide, has been increased by less than 5% during purification. More preferably, the content of these auto-degradation products has been increased by less than 4%, less than 3%, less than 2%, less than 1% during the purification step. More preferably, the content of these auto-degradation products has been increased by 0% during the purification step. In the most preferred embodiment of the present invention, the content of auto-degradation products has been reduced during the purification step.

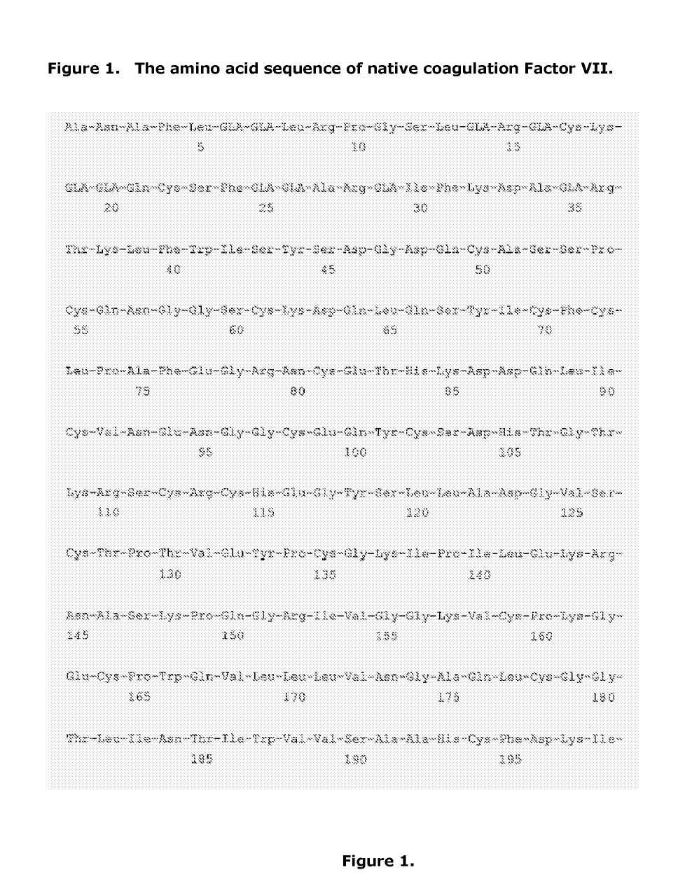

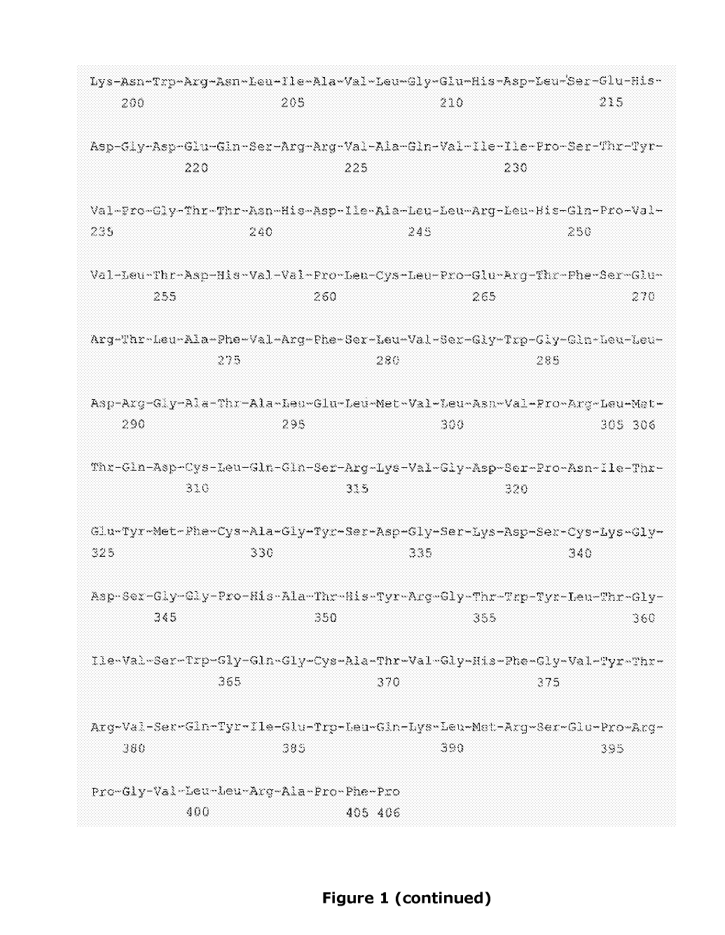

The positions 38, 290, and 315 refer to the positions in the human factor VII amino acid sequence as shown in FIG. 1. It will be understood that the corresponding cleavage sites (positions) in Factor VII sequence variants can easily be identified by sequence alignment.

The content of auto-degradation products may be measured by the method described in the present specification and is given in % of total area of all peaks in the chromatogram (e.g. as obtained by the method in Assay 5). The term “increasing the content of auto-degradation products by less than x % during purification” is intended to mean the that difference between the content of auto-degradation products prior to purification and after purification is at most x %. As an example, if the content of auto-degradation products prior to purification by the method of the present invention is 8% and it is increased by less than 5%, then the content after purification is at most 13%.

In another aspect the present invention provides a method for inhibiting Factor VIIa activity during the manufacture of Factor VII polypeptides (Factor VII or Factor VIIa) wherein a solution of a Factor VII or Factor VIIa has a temperature in the range from about 30° C. to about 45° C. In one embodiment the solution comprises an organic modifier in a concentration from about 2% w/w to about 40% w/w, e.g. ethanol.

The biological activity of Factor VIIa in blood clotting derives from its ability to (i) bind to Tissue Factor (TF) and (ii) catalyze the proteolytic cleavage of Factor IX or Factor X to produce activated Factor IX or X (Factor IXa or Xa, respectively).

The specific activity is expressed in International Units (IU) per mg FVIIa and it can be measured, e.g., by a clotting assay, where the biological activity of Factor VII polypeptides may be quantified by measuring the ability of a preparation to promote blood clotting, cf. Assay 4 described herein. In this assay, biological activity is expressed as the reduction in clotting time relative to a control sample and is converted to “Factor VII units” by comparison with a pooled human serum standard containing 1 unit/mL Factor VII activity. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X. (Persson et al., J. Biol. Chem. 272:19919-19924, 1997); (ii) measuring Factor X hydrolysis in an aqueous system (“In Vitro Proteolysis Assay”, see Assay 2 below); (iii) measuring the physical binding of Factor VIIa or a Factor VII-related polypeptide to TF using an instrument based on surface plasmon resonance (Persson, FEBS Letts. 413:359-363, 1997); (iv) measuring hydrolysis of a synthetic substrate by Factor VIIa and/or a Factor VII-related polypeptide (“In Vitro Hydrolysis Assay”, see Assay 1 below); or (v) measuring generation of thrombin in a TF-independent in vitro system (see Assay 3 below).

A wide range of FVII polypeptides can be purified using the method according to the pre-sent invention.

The term “Factor VII polypeptide” encompasses wild-type Factor VII (i.e. a polypeptide having the amino acid sequence disclosed in U.S. Pat. No. 4,784,950), variants thereof as well as Factor VII-related polypeptides, Factor VII derivatives and Factor VII conjugates, molecules with different number of GLA residues, molecules with a modified or incomplete glycosylation pattern. This includes FVII variants, Factor VII-related polypeptides, Factor VII derivatives and Factor VII conjugates exhibiting substantially the same or improved biological activity relative to wild-type human Factor VIIa.

The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. Such variants of Factor VII may exhibit different properties relative to human Factor VII, including stability, phospholipid binding, altered specific activity, and the like. “Factor VII” or “Factor VIIa” within the above definition also includes natural allelic variations that may exist and occur from one individual to another. Also, degree and location of glycosylation or other post-translation modifications may vary depending on the chosen host cells and the nature of the host cellular environment.

The term “Factor VII polypeptide” also encompasses polypeptides, including variants, in which the Factor VIIa biological activity has been substantially modified or somewhat reduced relative to the activity of wild-type Factor VIIa, but that typically retain a significant level of amino acid sequence identity to Factor VII, such as at least about 70%, at least about 75%, at least about 80%, at least about 85%, at least about 90%, at least about 93%, at least about 95%, or more (e.g., about 97, 98, or 99% identity). These polypeptides include, without limitation, Factor VII or Factor VIIa into which specific amino acid sequence alterations have been introduced that modify or disrupt the bioactivity of the polypeptide (as exemplified further herein).

The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof. Non-limiting examples of modifications of amino acid residues include amidation, alkylation, acylation and pegylation. Non-limiting examples of Factor VII derivatives includes GlycoPegylated FVII derivatives as disclosed in WO 03/31464 and US Patent applications US 20040043446, US 20040063911, US 20040142856, US 20040137557, and US 20040132640 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota); and FVII variants as disclosed in WO 01/58935, US patent U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

The term “increased-” or “improved biological activity” refers to Factor VII polypeptides with i) substantially the same or increased proteolytic activity compared to recombinant wild type human Factor VIIa or ii) to Factor VII polypeptides with substantially the same or increased TF binding activity compared to recombinant wild type human Factor VIIa or iii) to FVII polypeptides with substantially the same or increased half life in blood plasma compared to recombinant wild type human Factor VIIa. The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

Non-limiting examples of Factor VII variants having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. 3. Vasc. Endovasc. Surg. 15:515-520, 1998), and Factor VIIa lacking the Gla domain, (Nicolaisen et al., FEBS Letts. 317:245-249, 1993).

Examples of Factor VII polypeptides include, without limitation, wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys, and FVII having substitutions, deletions, or additions in the amino acid sequence Ile153-Arg223.

Thus, substitution variants in a Factor VII polypeptide include, without limitation substitutions in positions P10, K32, L305, M306, D309, L305, L305, F374, V158, M298, V158, E296, K337, M298, M298, S336, S314, K316, K316, F374, S52, S60, R152, S344, T106, K143, N145, V253, R290, A292, G291, R315, V317, and substitutions, additions or deletions in the amino acid sequence from T233 to N240 or from R304 to C329; or from 1153 to R223, or combinations thereof, in particular variants such as P10Q, K32E, L305V, M306D, D309S, L305I, L305T, F374P, V158T, M298Q, V158D, E296V, K337A, M298Q, M298K, S336G, S314E, K316H, K316Q, F374Y, S52A, S60A, R152E, S344A, T106N, K143N, N145T, V253N, R290N, A292T, G291N, R315N, V317T, P10Q/K32E, and substitutions, additions or deletions in the amino acid sequence from T233 to N240, or from R304 to C329, or from 1153 to R223, or combinations thereof.

Other Factor VII polypeptides which may be purified using the method according to the present invention are found in WO 01/83725, WO 02/22776 and WO 2004111242.

In some embodiments, the Factor VII polypeptide is human Factor VIIa (hFVIIa), preferably recombinantly made human Factor VIIa (rhFVIIa). In other embodiments, the Factor VII polypeptide is a Factor VII sequence variant. In some embodiments, the Factor VII polypeptide has a glycosylation different from wild-type human Factor VII.

In various embodiments, e.g. those where the Factor VII polypeptide is a Factor VII-related polypeptide or a Factor VII sequence variant, the ratio between the activity of the Factor VII polypeptide and the activity of native human Factor VIIa (wild-type FVIIa) is at least about 1.25, preferably at least about 2.0, or 4.0, most preferred at least about 8.0, when tested in the “In Vitro Proteolysis Assay” (Assay 2) as described in the present specification.

In some embodiments, the Factor VII polypeptides are Factor VII-related polypeptides, in particular variants, wherein the ratio between the activity of said Factor VII polypeptide and the activity of native human Factor VIIa (wild-type FVIIa) is at least about 1.25 when tested in the “In Vitro Hydrolysis Assay” (see Assay 1 below); in other embodiments, the ratio is at least about 2.0; in further embodiments, the ratio is at least about 4.0.

The terminology for amino acid substitutions used in this description is as follows. The first letter represents the amino acid naturally present at a position of the sequence in FIG. 1. The following number represents the position in the sequence in FIG. 1. The second letter represents the different amino acid substituting for the natural amino acid. An example is L305V/K337A-FVII, the leucine at position 305 of the sequence in FIG. 1 is replaced by a valine and the Lysine at position 337 of the sequence in FIG. 1 is replaced by an alanine, both mutations in the same FVII polypeptide variant.

The term “polypeptide product” as used herein means the purified peptide product which is to be used for the manufacture of a pharmaceutical composition. Thus, the polypeptide product is normally obtained as the product from the final purification, drying or conditioning step. The product may be crystals, precipitate, solution or suspension. The polypeptide product is also known in the art as the drug substance.

In a further aspect the present invention relates to a pharmaceutical composition pre-pared by admixing the polypeptide product produced according to the present invention with water for injection, an isotonicity agent and a calcium salt.

In one embodiment the pharmaceutical composition comprising a Factor VII polypeptide, contains less than 15% (w/w protein) auto-degradation products produced by cleavage at any of positions 38, 290, 315 or combinations thereof in Factor VII. In another embodiment the pharmaceutical composition comprising a Factor VII polypeptide contains less than 10% (w/w protein) auto-degradation products produced by cleavage at any of positions 38, 290, 315 or combinations thereof in Factor VII. In another embodiment the pharmaceutical composition comprising a Factor VII polypeptide contains less than 5% (w/w protein) auto-degradation products produced by cleavage at any of positions 38, 290, 315 or combinations thereof in Factor VII.

In another embodiment the pharmaceutical composition comprises at least one additional coagulation factor which is not FVII or FVIIa.

The content of degradation products can be determined by use of reverse phase HPLC as described by Mollerup et al. in Biotech & bioeng. 48, 1995, 501-505.

The term “pharmaceutical composition” as used herein means a product comprising a pharmaceutically active compound or a salt thereof together with pharmaceutical excipients such as buffer, tonicity modifier and optionally preservative and/or stabiliser. After collection of the fractions corresponding to the purified Factor VII, the polypeptide may be formulated into a solution, which may be dispensed into vials and freeze-dried. As an illustrative example of a final product corresponding to the commercially available, recombinantly-made FVII polypeptide composition NovoSeven® (Novo Nordisk A/S, Denmark) can be mentioned a vial (1.2 mg) containing 1.2 mg recombinant human Factor VIIa, 5.84 mg NaCl, 2.94 mg CaCl₂, 2H₂O, 2.64 mg GlyGly, 0.14 mg polysorbate 80, and 60.0 mg mannitol. This product is reconstituted to pH 5.5 by 2.0 mL water for injection (WFI) prior to use. When reconstituted, the protein solution is stable for use for 24 hours.

The overall manufacture of recombinant activated Factor VII (rFVIIa) is described by Jurlander, et al. in Seminars in Thrombosis and Hemostasis, Vol. 27, No. 4, 2001.

The pharmaceutical compositions are primarily intended for parenteral administration for prophylactic and/or therapeutic treatment. Preferably the pharmaceutical compositions are administered parenterally, i.e., intravenously, subcutaneously, or intramuscularly, or it may be administered by continuous or pulsatile infusion.

The compositions containing the Factor VII polypeptide variants of the present invention can be administered for prophylactic and/or therapeutic treatments. In therapeutic applications, compositions are administered to a subject already suffering from a disease, as described above, in an amount sufficient to cure, alleviate or partially arrest the disease and its complications. An amount adequate to accomplish this is defined as “therapeutically effective amount”. As will be understood by the person skilled in the art, amounts effective as a “therapeutically effective amount” will depend on the severity of the disease or injury as well as the weight and general state of the subject. In general, however, the effective amount will range from about 0.05 mg up to about 500 mg of the Factor VII polypeptide per day for a 70 kg subject, with dosages of from about 1.0 mg to about 200 mg of the Factor VIIa polypeptide per day being more commonly used.

The present invention also encompasses the use of the Factor VII compositions for one or more of: (a) the preparation of a medicament for the treatment of bleeding disorders or bleeding episodes or for the enhancement of the normal haemostatic system; or (b) the treatment of haemophiia A or B.

The present invention is further illustrated by the following examples which, however, are not to be construed as limiting the scope of protection. The features disclosed in the foregoing description and in the following examples may; both separately or in any combination thereof, be material for realising the invention in diverse forms thereof.

### EXAMPLES

### Assays Suitable for Determining Biological Activity of Factor Vii Polypeptides

Factor VII polypeptides useful in accordance with the present invention may be selected by suitable assays that can be performed as simple preliminary in vitro tests. Thus, the present specification discloses a simple test (entitled “In Vitro Hydrolysis Assay”) for the activity of Factor VII polypeptides.

### In Vitro Hydrolysis Assay (Assay 1)

Native (wild-type) Factor VIIa and Factor VII polypeptide (both hereinafter referred to as “Factor VIIa”) may be assayed for specific activities. They may also be assayed in parallel to directly compare their specific activities. The assay is carried out in a microtiter plate (MaxiSorp, Nunc, Denmark). The chromogenic substrate D-Ile-Pro-Arg-p-nitroanilide (S 2288; Chromogenix, Sweden); final concentration 1 mM, is added to Factor VIIa (final concentration 100 nM) in 50 mM HEPES, pH 7.4; containing 0.1 M NaCl, 5 mM CaCl2 and 1 mg,/mL bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used for calculating the ratio between the activities of Factor VII polypeptide and wildtype Factor VIIa:

Ratio=(A405 nm Factor VII polypeptide)/(A405 nm Factor VIIa wild-type).

Based thereon Factor VII polypeptides with an activity lower than, comparable to, or higher than native Factor VIIa may be identified, such as, for example, Factor VII poly peptides where the ratio between the activity of the Factor VII polypeptide and the activity of native Factor VII (wild-type FVII) is about 1.0 versus above 1.0. The activity of the Factor VII polypeptides may also be measured using a physiological substrate such as Factor X (“In Vitro Proteolysis Assay”), suitably at a concentration of 100-1000 nM, where the Factor Xa generated is measured after the addition of a suitable chromogenic substrate (eg. S-2765). In addition, the activity assay may be run at physiological temperature.

### In Vitro Proteolysis Assay (Assay 2)

Native (wild-type) Factor VIIa and Factor VII polypeptide (both hereinafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 μL 50 mM HEPES, pH 7.4, containing 0.1 M NaCl, 5 mM CaCl2 and 1 mg/mL bovine serum albumin, are incubated for 15 nm. Factor X cleavage is then stopped by the addition of 50 μL 50 mM HEPES, pH 7.4, containing 0.1 M NaCl, 20 mM EDTA and 1 mg/mL bovine serum albumin. The amount of Factor Xa generated is measured by the addition of the chromogenic substrate Z-D-Arg-Gly-Arg-p-nitroanilide (S-2765, Chromogenix, Sweden), final concentration 0.5 mM. The absorbance at 405 m/z is measured continuously in a SpectraMax™ 340 plate reader (Molecular Devices, USA). The absorbance developed during 10 minutes, after subtraction of the absorbance in a blank well containing no FVIIa, is used for calculating the ratio between the proteolytic activities of Factor VII polypeptide and wild-type Factor VIIa.

Ratio=(A405 nm Factor VII polypeptide)/(A405 nm Factor VIIa wild-type).

Based thereon, Factor VII polypeptide with an activity lower than, comparable to; or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

### Thrombin Generation Assay (Assay 3)

The ability of Factor VIIa or Factor VII polypeptides to generate thrombin can also be measured in an assay (Assay 3) comprising all relevant coagulation Factors and inhibitors at physiological concentrations (minus Factor VII when mimicking hemophilia A conditions) and activated platelets (as described on p. 543 in Monroe et al. (1997) Brt. J. Haematol. 99; S42-547, which is hereby incorporated herein as reference).

### One-Stage Coagulation Assay (Assay 4)

The biological activity of the Factor VII polypeptides may also be measured using a one stage coagulation assay (Assay 4). For this purpose, the sample to be tested is diluted in 50 mM PIPES buffer (pH 7.5), 0.1% BSA and 40 μl is incubated with 40 μl of Factor VII deficient plasma and 80 μl of human recombinant tissue factor containing 10 mM Ca2+ and synthetic phospholipids. Coagulation times are measured and compared to a standard curve using a reference standard in a parallel line assay.

### Determination of Heavy Chain Degradation Products and Purity of FVII Polypeptides (Assay 5)

The method previously described by Mollerup et. al (Biotechnology and Bioengineering, 1995, 48, 501-505) was used to detect and quantify the heavy chain degradation products in FVII solution:

RP-HPLC was performed on C4 substituted 300 Å silica produced in house. Column dimensions were 4×250 mm. Buffer A consisted of 0.1% TFA in water, buffer B of 0.09% TFA in 80% acetonitrile. Linear gradients were run with a flow rate of 1 mL/min at 70° C.

### Preparation and Purification of Factor VII Polypeptides

Human purified Factor VIIa suitable for use in the present invention is preferably made by DNA recombinant technology, e.g. as described by Hagen et al. Proc.Natl.Acad.Sci. USA 83: 2412-2416, 1986; or as described in European Patent No. 0 200 421 (ZymoGenetics, Inc.). The bovine FVII sequence is described in Takeya et al., 1. Biol. Chem. 263:14868-14872 (1988)).

Factor VII may also be produced by the methods described by Broze and Majerus, J. Biol. Chem. 255 (4): 1242-1247, 1980 and Hedner and Kisiel, J.Clin.Invest, 71: 1836-1841, 1983. These methods yield Factor VII without detectable amounts of other blood coagulation Factors. An even further purified Factor VII preparation may be obtained by including an additional gel filtration as the final purification step, Factor VII is then con verted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be completely activated by passing it through an ion-exchange chromatography column, such as Mono QC. (Pharmacia fine Chemicals) or the like, or by autoactivation in solution. FVII variants and VIII-related polypeptides may be produced by modification of wild-type FVII or by recombinant technology. FVII polypeptides with altered amino acid sequence when compared to wild-type FVII may be produced by modifying the nucleic acid sequence encoding wild-type FVII either by altering the amino acid codons or by removal of some of the amino acid codons in the nucleic acid encoding the natural FVII by known means, e.g. by site-specific mutagenesis.

It will be apparent to those skilled in the art that substitutions can be made outside the regions critical to the function of the FVIIa molecule and still result in an active polypeptide. Amino acid residues essential to the activity of the FVII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085) In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see; e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site directed mutagenesis using ant of the methods known in the art. Particularly useful is the procedure that utilizes a super coiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated, Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used; such as, for example, gene shuffling or phage display techniques.

The nucleic acid construct encoding the FVII polypeptide of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd. Ed. Cold Spring Harbor Laboratory, Cold Spring Harbor, N.Y., 1989). The nucleic acid construct encoding the FVII polypeptide of interest may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques. The nucleic acid construct is preferably a DNA construct. Transgenic animal technology may be employed to produce the FVII or FVIIa variants of the invention, e.g., within the mammary glands of a host female mammal (see, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49: 113-120 (1992); U.S. Pat. Nos. 4,873,191; 4,873,316; WO 88/00239, WO 90/05188, WO 92/11757; and GB 87/00458).

Production in transgenic plants may also be employed. Expression may be generalised or directed to a particular organ, such as a tuber (see, Hiatt, Nature 344:469-479 (1990); Edelbaum et al., J. Interferon Res. 12:449-453 (1992); Sijmons et al., Bio/Technology 8:217-221 (1990); and EP 0 255 378).

Separation of polypeptides from their cell of origin may be achieved by any method known in the art, including, without limitation, removal of cell culture medium containing the desired product from an adherent cell culture; centrifugation or filtration to remove non-adherent cells, and the like. The elevated temperatures according to the present invention may be applied to a number of these purification steps. However, it is expected that the need to lower the activity of FVIIa increases with the purity and concentration of the Factor VII polypeptide.

Following purification, the preparation of the polypeptide product preferably contains less than about 15% by weight, more preferably less than about 10%, more preferred less than about 5%, and most preferably less than about 1%, of non-FVII proteins derived from the host cell.

If needed, Factor VII polypeptides may be activated by proteolytic cleavage, using Factor XIIa or other proteases having trypsin-like specificity, such as, e.g. Factor IXa, kallikrein, Factor Xa, and thrombin. See, e.g. Osterud et al. Biochenm. 11:2853 (1972); Thomas, U.S. Pat. No. 4,456,591; and Hedner et al. 3. Clin. Invest. 71:1836 (1983A), Alternatively, Factor VII polypeptides may be activated by passing it through an ion-exchange chromatography column, such as Mono QC (Pharmacia) or the like, or by auto-activation in solution. The resulting activated Factor VII polypeptide may then be formulated and administered as described in the present application.

The following examples illustrate practice of the processes of the invention. These examples are included for illustrative purposes only and are not intended in any way to limit the scope of the invention claimed.

### Examples

### AIEC of FVII at 40° C. and 5° C.

For binding of FVII to anion exchange resin it is necessary to remove divalent cations such as Ca²⁺ from the solution of FVII by chelating using metal chelating agents such as EDTA prior to loading

AEIC was performed at flow rate of 40 CV/h

### Example 1

### Performing AIEC at 40° C., DH 8.6

A solution of FVII containing 14.4 mg FVII with a specific activity of 60000 IU/mg was loaded onto a Q Sepharose FF column (CV: 1 mL) equilibrated with 175 mM NaCl and 10 mM glycylglycine, pH 8.6. After washing with the equilibration buffer (3 CV), the column was washed with 50 mM NaCl and 10 mM glycylglycine, pH 8.6 (2CV), and subsequently elution was performed with step gradient with 50 mM NaCl, 15 mM CaCl₂, 10 mM glycylglycine, pH 8.6. The fractions containing FVII were pooled and analysed by RP-HPLC (assay 5) and clot assay (assay 4). Yield: 51%, specific activity (52230 IU/mg)

### Example 2

### Performing AIEC at 40° C., DH 6.0

A solution of FVII containing 14.4 mg FVII with a specific activity of 60000 IU/mg was loaded onto a Q Sepharose FF column (CV: 1 mL) equilibrated with 175 mM NaCl and 10 mM histidine, pH 6.0. After washing with the equilibration buffer (3 CV), the column was washed with 50 mM NaCl and 10 mM histidine, pH 6.0 (2CV), and subsequently elution was performed with step gradient with 50 mM NaCl, 35 mM CaCl₂, 10 mM histidine, pH 6.0. The fractions containing FVII were pooled and analysed by RP-HPLC (assay 5) and clot assay (assay 4). Yield: 56%, specific activity (56600 IU/mg)

### Example 3

### Performing AIEC at 40° C., DH 8.6, and Arginine in Elution Buffer

A solution of FVII containing 14.4 mg FVII with a specific activity of 60000 IU/mg was loaded onto a Q Sepharose FF column (CV: 1 mL) equilibrated with 175 mM NaCl and 10 mM glycylglycine, pH 8.6. After washing with the equilibration buffer (3 CV), the column was washed with 50 mM NaCl and 10 mM glycylglycine, pH 8.6 (2 CV), and subsequently elution was performed with step gradient with 200 mM NaCl, 35 mM CaCl₂, 10 mM glycylglycine, 1 M arginine, pH 8.6. The fractions containing FVII were pooled and analysed by RP-HPLC (assay 5) and clot assay (assay 4). Yield 52%, specific activity (49714 IU/mg).

### Example 4

### Performing AIEC at 40° C., DH 8.6, and Arginine in Fraction Tubes

A solution of FVII containing 14.4 mg FVII with a specific activity of 60000 IU/mg was loaded onto a Q Sepharose FF column (CV: 1 mL) equilibrated with 175 mM NaCl and 10 mM Tris, pH 8.6. After washing with the equilibration buffer (3 CV), the column was washed with 50 mM NaCl and 10 mM Tris, pH 8.6 (2 CV), and subsequently elution was performed with step gradient with 175 mM NaCl, 35 mM CaCl₂, 10 mM Tris, pH 8.6 into fraction tubes containing 1 M arginine. FVII containing fractions were pooled and analysed by RP-HPLC (assay 5) and clot assay (assay 4). Yield 37%, specific activity (52100 IU/mg).

### Example 5

### Performing AIEC at 5° C., DH 8.6

A solution of FVII containing 28 mg FVII with a specific activity of 60000 IU/mg was loaded onto a Q Sepharose FF column (CV: 2.2 mL) equilibrated with 175 mM NaCl and 10 mM Tris, pH 8.6. After washing with the equilibration buffer (3 CV), the column was washed with 50 mM NaCl and 10 mM Tris, pH 8.6 (2 CV), and subsequently elution was performed with step gradient with 200 mM NaCl, 35 mM CaCl₂, 10 mM Tris, pH 8.6. FVII containing fractions were pooled and analysed by RP-HPLC (assay 5) and clot assay (assay 4). Yield 46%, specific activity (58830 IU/mg).

TABLE 1
Results from RP-HPLC analysis (assay 5) and Clot assay (assay 4).
290- and 315-impurities are obtained by cleavage at amino acid
290 and 315, respectively. Total is the sum of the 219 and 315
impurities. The results show that AIEC of activated FVII give less
auto-degradation products at 40° C. than at 5° C. The lowest value
obtained for specific activity is about 82% (from example 3) of the
specific activity of FVII in the application.

| | Temp | Yield | 290- | 315- | | Specific |
|---|---|---|---|---|---|---|
| Sample | (° C.) | (%) | impurity | impurity | Total | activity |
| Application | | | 2.4 | 8.4 | 10.8 | 60000 |
| Example 1 | 40 | 51 | 1.0 | 7.8 | 8.8 | 52230 |
| Example 2 | 40 | 56 | 1.8 | 8.3 | 10.1 | 56600 |
| Example 3 | 40 | 52 | 0.7 | 7.3 | 7.9 | 49714 |
| Example 4 | 40 | 37 | 1.0 | 6.5 | 7.5 | 52100 |
| Example 5 | 5 | 46 | 2.9 | 9.7 | 12.6 | 58830 |

HIC of FVII at 37° C. and RT

HIC was performed at flow rate of 30 CV/h

### Example 6

### HIC at 37° C.

A solution of FVII containing 10 mg FVII with a specific activity of 49901 IU/mg was loaded onto a TSK-phenyl 5 PW column (CV: 5 mL) equilibrated with 1.8 M ammonium acetate and 20 mM histidine, pH 8.6. After washing with the equilibration buffer (3 CV), the column was eluted with gradient over 18 CV with 50 mM ammonium acetate and 20 mM histidine, pH 8.6. FVII containing fractions were pooled and analysed by RP-HPLC (assay 5) and clot assay (assay 4). Yield 77%, specific activity (47368 IU/mg).

### Example 7

### HIC at RT

A solution of FVII containing 10 mg FVII with a specific activity of 49901 IU/mg was loaded onto a TSK-phenyl 5 PW column (CV: 5 mL) equilibrated with 1.8 M ammonium acetate and 20 mM histidine, pH 8.6. After washing with the equilibration buffer (3 CV), the column was eluted with gradient over 18 CV with 50 mM ammonium acetate and 20 mM histidine, pH 8.6. FVII containing fractions were pooled and analysed by RP-HPLC (assay 5) and clot assay (assay 4). Yield 88%, specific activity (51678 IU/mg).

TABLE 2
Results from RP-HPLC analysis (assay 5) and Clot assay (assay 4).
290- and 315-impurities are obtained by cleavage at amino acid
290 and 315, respectively. Total is the sum of the 219 and 315
impurities. The results show that HIC of activated FVII give less
auto-degradation products at 37° C. than at RT. The lowest value
obtained for specific activity is about 95% (from example 6) of the
specific activity of FVII in the application.

| | Temp | Yield | 290- | 315- | | Specific |
|---|---|---|---|---|---|---|
| Sample | (° C.) | (%) | impurity | impurity | Total | activity |
| Application | | | 2.2 | 5.8 | 8.0 | 49901 |
| Example 6 | 37 | 77 | 0.5 | 5.0 | 5.5 | 47368 |
| Example 7 | RT | 88 | 2.3 | 6.2 | 8.5 | 51678 |

1. A method for purification of a Factor VII polypeptide wherein the temperature during purification is maintained in the range from 30° C. to 45° C.

2. The method according to claim 1, wherein said temperature is in the range from 35° C. to 40° C.

3.-5. (canceled)

6. The method according to claim 1, wherein (i) the resulting Factor VII polypeptide retains at least 30% of its specific activity and/or (ii) the content of auto-degradation products produced by cleavage at one or more of positions 38, 290, and 315, in said Factor VII polypeptide has been increased by less than 5% during the purification step.

7. (canceled)

8. The method according to claim 1, wherein purification of said Factor VII polypeptide is performed in an aqueous solution comprising an organic modifier.

9. The method according to claim 8, wherein said organic modifier is selected from the group consisting of: ethanol, 1-propanol, 2-propanol, acetonitrile, hexylene glycol, and mixtures thereof.

10. The method according to claim 8, wherein the concentration of said organic modifier is from 2% w/w to 40% w/w.

11. The method according to claim 1, wherein at least one protein stabilizer is used to stabilise the Factor VII polypeptide.

12. The method according to claim 11, wherein the protein stabilizer is selected from the group consisting of a sugar, an amino acid, or combinations thereof.

13. (canceled)

14. The method according to claim 12, wherein the protein stabilizer is arginine in a concentration in the range from 0.5 M to 5 M.

15. The method according to claim 1, wherein a refolding agent is used.

16. The method according to claim 15, wherein said refolding agent is ethylene glycol in the range from 0.5 M to 10 M.

17. (canceled)

18. The method according to claim 1, wherein said purification is chromatographic purification or membrane purification.

19-21. (canceled)

22. The method according to claim 18, wherein said chromatographic purification is selected from the group consisting of hydrophobic interaction chromatography, size exclusion chromatography, ion exchange chromatography, and affinity chromatography.

23-32. (canceled)

33. A method according to claim 1, wherein said temperature is in the range from 35° C. to 45° C.

34-35. (canceled)

36. A method according to claim 1, wherein said Factor VII polypeptide is selected from the group consisting of wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, and K316Q/L305V/V158T/E296V/M298Q/K337A-FVII, and PEGylated forms hereof wherein the PEG group or groups is/are attached to the protein backbone of the Factor VII polypeptide and/or attached to a carbohydrate moiety of the Factor VII polypeptide.

37. A method for inhibiting Factor VIIa activity during the manufacture of Factor VII polypeptides, wherein a solution of said Factor VII polypeptides has a temperature in the range from 30° C. to 45° C.

38. A method according to claim 37, wherein said solution comprises an organic modifier in a concentration from 2% w/w to 40% w/w.
